(19) United States
(12) Patent Application Publication
(10) Pub. No.: US 2010/0174294 A1
(43) Pub. Date: Jul. 8, 2010

(54) ELIMINATION-EQUIPMENT OF HORNY SUBSTANCE TO THE SOLE OF A FOOT

(76) Inventors: Do A. Kim, Daegu (KR); Seong Cheol Han, Daegu (KR)

Correspondence Address: PARK LAW FIRM, 3255 WILSHIRE BLVD, SUITE 1110, LOS ANGELES, CA, 90010, US

(21) Appl. No.: 12/602,393

(22) PCT Filed: May 30, 2008
(86) PCT No.: PCT/KR08/03040

§ 371 Date: Nov. 30, 2009

(30) Foreign Application Priority Data
Jun. 1, 2007 (KR) 10-2007-0053987

(51) Int. Cl. A61B 17/54

(52) U.S. Cl. 606/131

(57) ABSTRACT

An apparatus of effectively and easily eliminating horny substances on the sole of a foot that has the most severe symptom of horny substance among various parts of a human body is provided. That is, the invention is directed to an apparatus of removing horny substances, which includes: an electric motor M disposed at a lower position of the apparatus; a horny substance polishing member 10 shaped at an axis of the electric motor by means of injection molding using a synthetic resin, the horny substance polishing member 10 having at its top surface a polishing surface 11 with a constant curvature for making it easy to rub the palm of the hand or the sole of the foot after having the palm or sole in contact with the top surface; a polishing material 20 having particles of 60 to 120 mesh size, the particles being mixed with at least one selected from the group consisting of alumina, silicon carbide, emery, garnet and silica powder and uniformly secured to the polishing surface 11; and a water spraying device 30 disposed at the other end of the polishing member 10 and spraying a proper amount of water to the palm of the hand or the sole of the foot for removing the horny substance.

### TECHNICAL FIELD

The present invention relates to an apparatus of effectively and easily eliminating horny substances on the sole of a foot that has the most severe symptom of horny substance among various parts of a human body.

### BACKGROUND ART

Horny substances are typically formed of keratin (i.e., protein of a structure such as skin) that is generated by itself per a constant period to protect skins from outer environment and are formed all skins of a human body, however, cause a thicker horny substance layer to be generated on positions such as the sole of the foot or the palm of the hand where a degree of impact is high to protect the skin from the impact, which is also referred to as a hardened skin or a dead skin.

Such horny substances are generated in the skin by themselves to protect the skin from various external environments so that constant and normal horny substances are necessary for the human body.

However, the dead skin, which has lost its function (i.e., skin protecting function) after a predetermined time passes from its generation, and the old or excessively thick horny substances block skin pores to be harmful to skin. In addition, remaining dead substances may fall a prey to bacteria to help propagation of the bacteria which in turn may cause skin troubles, athlete's foot, etc.

Further, thick dead skins may be cracked to cause a high possibility of severe pains and infiltration of the bacteria, so that it is required to remove the dead skins to the extent of not damaging the skins.

To cope with this problem, a conventional method of removing the horny substance allows the foot to be soaked in water for a long time and removes the horny substance from the heel of the foot by the hand holding a towel or stone with a rough surface at the time of taking a bath, or uses a sharp blade to remove the thick and hardened skin by scratching or cutting. In recent times, devices of removing horny substances or electric motor type devices of removing horny substances using a high speed rotation type motor are put into market one after another so that its use is significantly increasing.

However, it is actually difficult to remove the old or dead skin by means of these conventional devices, which may rather damage the normal skin. In addition, it is not easy for the hand far from the sole of the foot to rub the sole, and above all things, the conventional manual-type or electric motor-type device of removing the horny substance uses objects (e.g. stone having a rough surface, a kind of a polishing material, a particle size, etc.) which does not take into consideration of unique properties of the horny substance eve if the substance is dead, so that a high efficiency of removing the hardened substance can not be expected, which thus requires the continuous improvement thereon.

In the meantime, the horny substances are keratins that are hardened on the outer skin of the human body, and may be different some or less depending on the dry skin type or oily skin type. The horny substance becomes soft when it is in contact with water or soaked in water and is then so hardened when it is dry. Among these states, the horny substances, when in a soft state due to water, are not polished even by a polishing stone, so that it may be more effective to take away or pull the horny substance that has been soaked for a very long time by rubbing the outer skin with a stone having a very rough surface. However, when the horny substance is dry and very hardened, it is the most idealistic way to polish the outer skin using a polishing stone, which causes fine particles of the polished substances to be attached between the polishing particles to make the rough polishing surface blunt, so that it becomes difficult to carrying out effective polishing.

Accordingly, even if the conventional methods as described above and other various methods or apparatus for removing the horny substance are employed, it is not expected to achieve a satisfactory efficiency.

### DISCLOSURE OF INVENTION

### Technical Problem

In order to solve the foregoing and/or other problems, it is an objective of the present invention to provide a novel apparatus of effectively removing horny substances, which first recognizes properties of the horny substance attached to a polishing surface different from a typical solid which cannot be polished when it is soft due to a contact with water but can be polished when it is dry, establishes new physical conditions of the horny substance that allows polishing, and establishes an optimal condition for polishing based on the new physical conditions.

### Technical Solution

In one aspect, the invention is directed to an apparatus of removing horny substances, which includes: an electric motor M disposed at a lower position of the apparatus; a horny substance polishing member 10 shaped at an axis of the electric motor by means of injection molding using a synthetic resin, the horny substance polishing member 10 having at its top surface a polishing surface 11 with a constant curvature for making it easy to rub the palm of the hand or the sole of the foot after having the palm or sole in contact with the top surface; a polishing material 20 having particles of 60 to 120 mesh size, the particles being mixed with at least one selected from the group consisting of alumina, silicon carbide, emery, garnet and silica powder and uniformly secured to the polishing surface 11; and a water spraying device 30 disposed at the other end of the polishing member 10 and spraying a proper amount of water to the palm of the hand or the sole of the foot for removing the horny substance.

### Advantageous Effects

According to the apparatus and method of the present invention as mentioned above, horny substances of the palm of the hand or the sole of the foot which has fundamentally different property from a typical solid can be effectively and easily polished and removed at the time of polishing the horny substance.

### BRIEF DESCRIPTION OF THE DRAWINGS

The foregoing and other objects and advantages of the invention will be apparent from the more particular description of preferred embodiments of the invention, as illustrated in the accompanying drawings. The drawings are not necessarily to scale, emphasis instead being placed upon illustrating the principles of the invention.

FIG. 1 is a perspective diagram according to a preferred embodiment of the present invention.

FIG. 2 is a disassembled perspective diagram of main parts according to the present invention.

FIG. 3 is an enlarged cross-sectional diagram illustrating a polishing surface on a polishing member and a polishing material to be applied and secured to the polishing surface according to the present invention.

FIGS. 4 and 5 are diagrams according to other embodiments of the present invention.

FIG. 6 is a diagram illustrating water spraying according to the present invention.

### BEST MODE FOR CARRYING OUT THE INVENTION

The present invention provides to an apparatus of removing horny substances, which includes: an electric motor M disposed at a lower position of the apparatus; a horny substance polishing member 10 shaped at an axis of the electric motor by means of injection molding using a synthetic resin, the horny substance polishing member 10 having at its top surface a polishing surface 11 with a constant curvature for making it easy to rub the palm of the hand or the sole of the foot after having the palm or sole in contact with the top surface; a polishing material 20 having particles of 60 to 120 mesh size, the particles being mixed with at least one selected from the group consisting of alumina, silicon carbide, emery, garnet and silica powder and uniformly secured to the polishing surface 11; and a water spraying device 30 disposed at the other end of the polishing member 10 and spraying a proper amount of water to the palm of the hand or the sole of the foot for removing the horny substance.

### Mode for the Invention

Hereinafter, the present invention will be described with reference to the accompanying drawings.

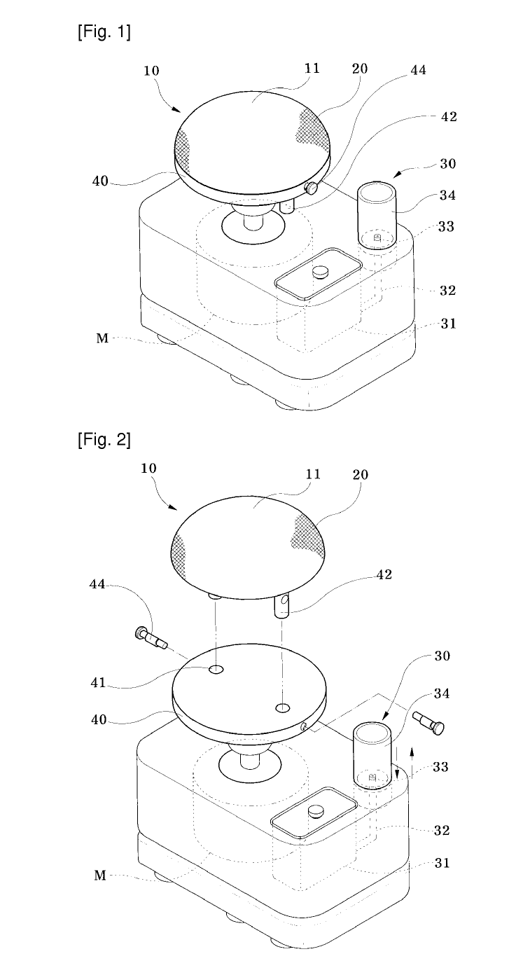

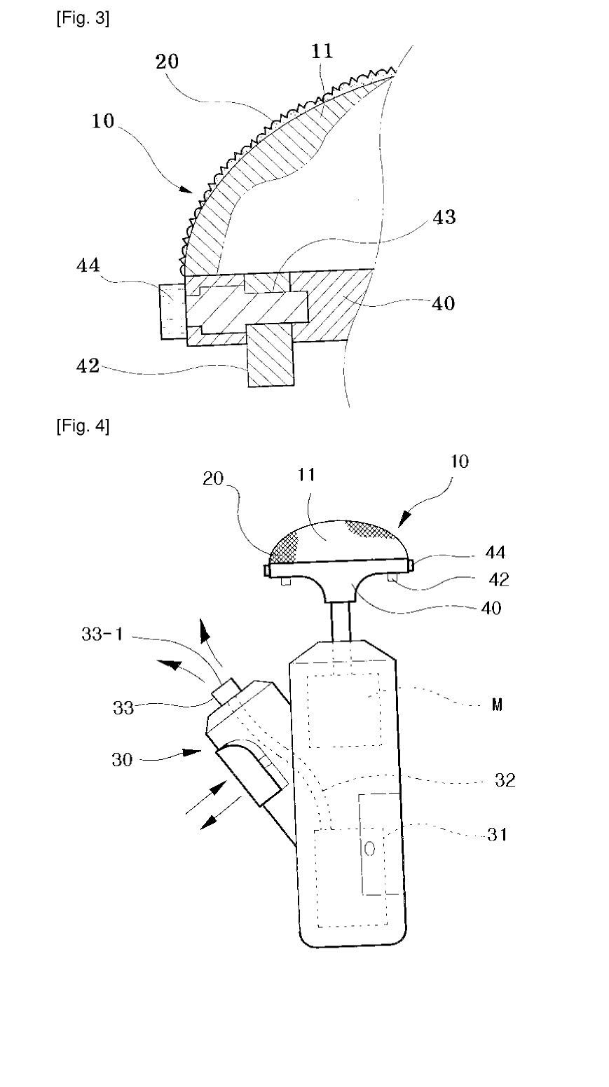

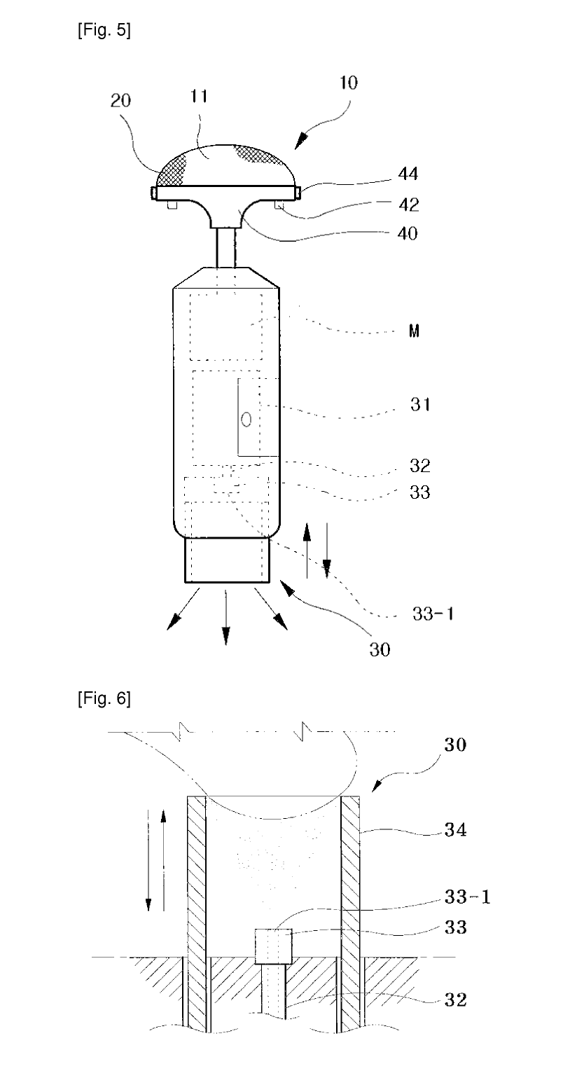

The present invention is characterized in that it includes, as shown in FIG. 1, an electric motor M disposed at a lower position of the apparatus; a horny substance polishing member 10 shaped at an axis of the electric motor by means of injection molding using a synthetic resin, allowing the horny substance to be removed by rubbing, and having at its top surface a polishing surface 11 with a constant curvature for making it easy to rub the palm of the hand or the sole of the foot after having the palm or sole in contact with the top surface; a polishing material 20 having particles of 60 to 120 mesh size, the particles being mixed with at least one selected from the group consisting of alumina, silicon carbide, emery, garnet and silica powder and uniformly secured to the polishing surface 11; and a water spraying device 30 disposed at the other end of the polishing member 10 and spraying a proper amount of water to the palm of the hand or the sole of the foot for removing the horny substance.

Next, according to a method of removing the horny substance of the present invention, a manual-type or electric motor-type apparatus of removing the horny substance is employed, wherein a polishing material 20 having particles of 60 to 120 mesh size which is mixed with at least one selected from the group consisting of alumina, silicon carbide, emery, garnet and silica powder is secured to a polishing surface 11, and a small amount of moisture that does not allow dropping of the moisture is sprayed to the hand or sole of the foot where the horny substance needs to be removed to make the horny substance soft, the polishing member 10 is then closely attached to the hand or sole and is rotated. At this time, a rotating speed of the polishing member 10 is made slow to a degree of 60 to 180 rpm to make the polishing efficiency as much as possible. When the moisture contained in the horny substance becomes dry during the polishing, the moisture, a small amount of the moisture that does not allow dropping is sprayed again to keep the horny substance soft while the polishing is carried out.

The polishing material 20 is formed of at least one selected from the group consisting of alumina, silicon carbide, emery, garnet and silica powder and secured to the polishing surface 11, which thus increases the cutting efficiency with respect to the horny substance as much as possible and guarantees a long lifetime due to the decrease in bluntness of the rough polishing particles even in a long-time use. The particle size of the polishing material 20 is set to 60 to 120 mesh, which is particularly the range having the highest cutting efficiency with respect to the thick horny substance formed on the hand or the sole of the foot. When the particle size is thick enough to fall below 60 mesh, the particle is so rough so that it may damage the skin. When the particle size is small enough to exceed 120 mesh, cutting is hardly carried out so that the efficiency of removing the horny substance is hardly shown.

Before the substantial polishing, a small amount of moisture that does not allow dropping is sprayed to the hand or sole of the foot where the horny substance needs to be removed. When the amount becomes excessive to cause dropping, the horny substance is excessively soft to make it difficult to carry out polishing. When the moisture is not sprayed to the horny substance at all, the horny substance becomes hardened to cause powers of the horny substance to be attached to the polishing surface 11, which makes it more difficult to carry out polishing. Accordingly, it is very important and necessary for the method of removing the horny substance to spray the small amount of moisture that does not allow dropping while the powders are not easily attached to the polishing surface 11.

In addition, it is very important to set the rotating speed of the polishing member 10 to a very slow speed such as 60 to 180 rpm when the polishing is carried out under the conditions described above. That is, in consideration of the particle size of the polishing material 20 such as 60 to 120 mesh and friction and cutting rate with the horny substance with respect to the particle size, when the speed exceeds 180 rpm, the horny substance is rapidly cut and polished so that the user cannot expect or properly cope with the situation to significantly damage the skin. When the speed falls below 60 rpm, removal of the horny substance is not easily carried out.

That is, according to the polishing on the horny substance as described above, the polishing surface 11 of the polishing member 10 uses the polishing material 20 mixed with at least one selected from the group consisting of alumina, silicon carbide, emery, garnet and silica powder, the particle size is in a range of 60 to 120 mesh, the small amount of moisture that does not allow dropping is sprayed and contained to the hand or sole of the foot where the horny substance needs to be removed, and the rotating speed of the polishing member 10 at this time is set to a slow speed such as 60 to 180 rpm. As such, if any one of the conditions mentioned above is not met, it is actually difficult to effectively remove the horny substance.

In the meantime, the apparatus for achieving the method has the electric motor M as described above. This electric motor M may be a deceleration motor in which a deceleration gear having a required speed ratio is built in. However, an inverter electric motor may also be employed which has an inverter having a speed adjustment volume built in an AC motor to freely adjust/control the rotating speed of the motor by means of constant frequency adjustment.

In addition, the polishing member 10 having the polishing surface 11 needs to be easily attached or detached for one s own use because a user may have a feeling of rejection when he uses the same polishing member 10 as another user has used before. Accordingly, the rotating plate 40 is disposed at the motor axis in a position where the axis of the motor M and the polishing member 10 are connected to allow the polishing member 10 to be closely attached to the position, at least one connection hole 41 is disposed in the rotating plate 40 while a connection pin 42 is formed in the polishing member 10 so that the connection pin is inserted and connected to the connection hole in the polishing member 10, and a sticking pin 44 that can be inserted into the sticking pin groove 43 and then stuck by half-turning is disposed to securely hold the connection pin 42 at the side end of the connection hole of the rotating plate 40.

The water spraying device 30 may have a general structure such that a spraying lever is operated by a finger for simplicity of use. However, it may have the rectangular or cylindrical water spraying member 34 disposed at a separate position, which carries out piston movement in an up or down direction and allows water contained in the water container 31 to be sprayed through the hose 32 and the spraying nozzle 33 by a small amount when a user puts his hand or foot on the water spraying member and pushes the water spraying member or contacts his hand or foot with the water spraying member and allows pumping to be carried out by a predetermined width. The water spraying member 34 may be connected to a piston for pumping disposed at a lower position of the water spraying member 34 so that a predetermined amount of water may be sprayed according to the pumping of the water spraying member 34, and such a principle of spraying the water is the same as the principle of water spraying device that may be easily found in our daily life, so that a more detailed description and drawings thereof will be omitted for simplicity of description.

The water spraying nozzle 33 preferably has a nozzle groove 33-1 with a very small bore so as to allow a small amount of moisture to be sprayed like a mist and contained in the surface of the horny substance.

In addition, the apparatus of removing the horny substance according to the present invention may have a separate power line and a power supply plug for supplying a power to the motor M, and may have a rechargeable electrode if necessary, and may have a battery (a disposable battery, a rechargeable battery, etc.) built in as required (not shown).

Preferred embodiments of the present invention have been disclosed herein and, although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

1. An apparatus of removing horny substances comprising:
an electric motor M disposed at a lower position of the apparatus;
a horny substance polishing member 10 shaped at an axis of the electric motor by means of injection molding using a synthetic resin, the horny substance polishing member 10 having at its top surface a polishing surface 11 with a constant curvature for making it easy to rub the palm of the hand or the sole of the foot after having the palm or sole in contact with the top surface;
a polishing material 20 having particles of 60 to 120 mesh size, the particles being mixed with at least one selected from the group consisting of alumina, silicon carbide, emery, garnet and silica powder and uniformly secured to the polishing surface 11; and
a water spraying device 30 disposed at the other end of the polishing member 10 and spraying a proper amount of water to the palm of the hand or the sole of the foot for removing the horny substance.

2. The apparatus according to claim 1, further comprising:
a rotating plate 40 having at least one connection hole 41, being disposed at the motor axis in a position where the axis of the motor M and the polishing member 10 are connected, and allowing the polishing member 10 to be closely attached to;
a connection pin 42 disposed at the polishing member 10 for being inserted and connected to the connection hole 41; and
a sticking pin 44 that is disposed at a side end of the rotating plate 40 and can be inserted to a sticking pin groove 43 and half-turned for securely holding the connection pin 42.

3. The apparatus according to claim 1, wherein the water spraying device 30 includes a water container 31, a hose 32 and a water spraying member 34, and the water spraying member has a rectangular or cylindrical shape that allows water contained in the water container 31 to be sprayed within a predetermined closed space through the hose 32 and a spraying nozzle 33 by a small amount when a user put his hand or foot on the water spraying member and pushes the water spraying member or contacts his hand or foot with the water spraying member and allows pumping to be carried out by a predetermined width.
